(19) United States
(12) Plant Patent Application Publication
(10) Pub. No.: US 2005/0166296 P1
(43) Pub. Date: Jul. 28, 2005

(54) Dendrobium plant named 'WHITE ANNA'

(50) DENDROBIUM HYBRIDA; DENDROBIUM

(76) Inventor: Surapon Sanguthai, Nakhonphatom (TH)

Correspondence Address: SURAPON SANGUTHAI;BANGKOK GREEN CO., LTD., 42 MOO 6, BUDTHAMONTHOL 4TH RD., SAMPRAN, NAKHONPHATOM, 73220, TH

(21) Appl. No.: 10/769,689

(22) Filed: Jan. 26, 2004

(51) Int. Cl. C12Q 1/68; A01H 5/00

(52) U.S. Cl. PLT/311

(57) ABSTRACT

A new and distinct cultivar of Dendrobium named ‘White Anna’ characterized by long lasting, round-shape white flowers on a long inflorescence.

### BACKGROUND OF THE INVENTION

The present invention is directed to a new and distinct cultivar of Dendrobium, botanically know as Dendrobium hybrid, and having the cultivar name ‘WHITE ANNA’.

The new cultivar was an elite somaclonal variant of Dendrobium hybrid ‘Anna Bride’ (Dendrobium Ekapol ‘Anna’×Dendrobium Shavin White ‘5N’) selected, from approximately 10,000 micropropagated plants of the original cultivar grown for cut—flower production, by the inventor.

Asexual propagation of the new cultivar in Nonthaburi, Thailand by division and micropropagation has demonstrated the uniformity and stability througout successive generations of this new Dendrobium.

### SUMMARY OF THE INVENTION

The following traits have been repeatedly recorded in the new cultivar grown under 50% shade condition of a commercial cut flower nursery in Nonthaburi, Thailand with common horticultural practice.

The features that in combination are believed to distinguish ‘White Anna’ from other known cultivars of Dendrobium include : color of petals, sepals and labellum; round-shape flowers; long postharvest life; long inflorescence.

Plants of the new Dendrobium can be compared to plants of its original cultivar, the Dendrobium hybrid ‘Anna Bride’ (patent apply—for, Docket number 2546/BGD/SW581). Plants of the new Dendrobium differ from plants of the cultivar ‘Anna Bride’ which is the most resemblance, in the following characteristics

1. The flower of the new Dendrobium is white, instead of light purplish pink with white center as found in ‘Anna Bride’.

The new cultivar has white petals, sepals and labellum whilst its female parent, D. Ekapol ‘Anna’, has pink petals, sepals and labellum. On the other hand, the flowers of the new cultivar has broader petals and rounder shape than its male parent, D. Shavin White ‘5N’.

### BRIEF DESCRIPTION OF ILLUSTRATIONS

The accompanying color photographs illustrate the overall appearance of the new cultivar as true in color as possible in color illustration of this type.

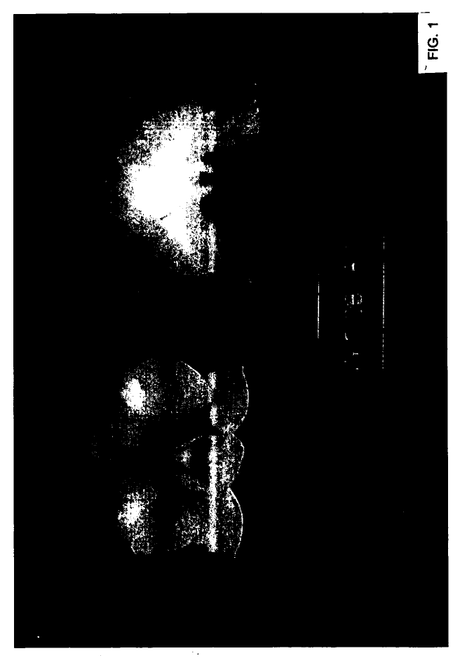

FIG. 1 is a front and rear view of Dendrobium hybrid ‘White Anna’.

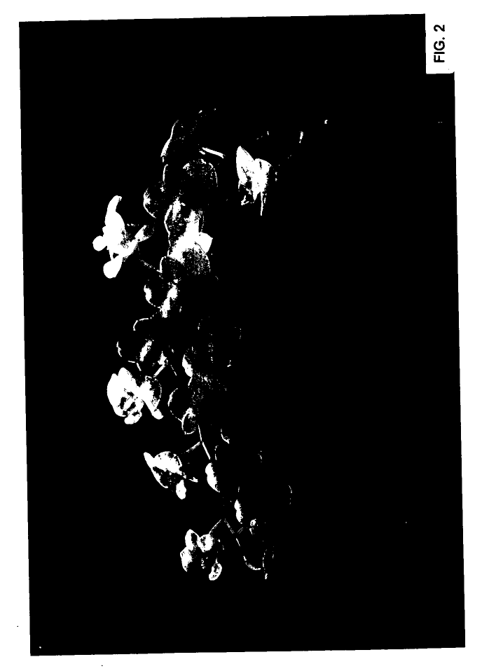

FIG. 2 is an inflorescence of Dendrobium hybrid ‘White Anna’.

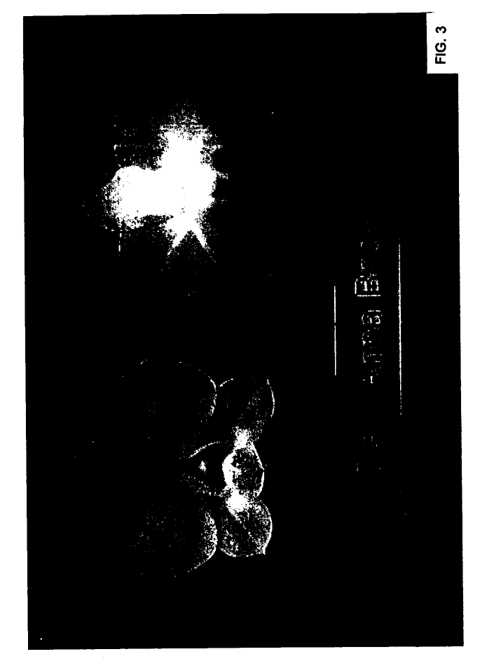

FIG. 3 is a front view of Dendrobium hybrid ‘Anna Bride’.

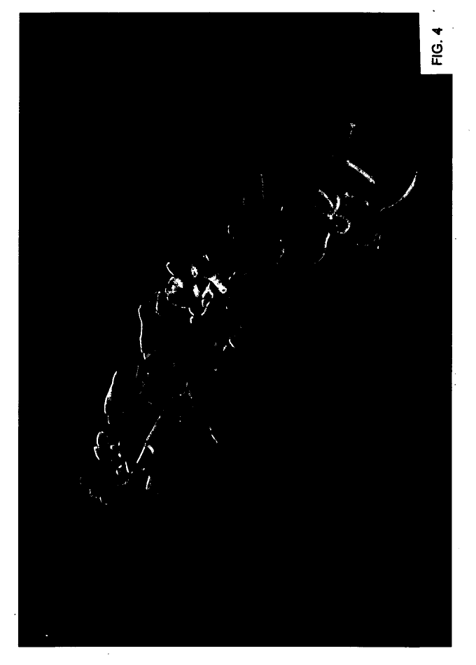

FIG. 4 is an inflorescence of Dendrobium hybrid ‘Anna Bride’.

### DESCRIPTION OF THE NEW VARIETY

The following observations were made of 3-year old plants of the new variety grown under light shade conditions, in Nonthaburi, Thailand during the months of March through August. Phenotypic expression may vary with environmental, cultural and climatic conditions, as well as differences in conditions of light and medium.

Throughout this specification, color references and/or values are based upon The Royal Horticultural Society Colour Chart (2001 edition) and terminology used in the color descriptions herein refers to plate number in this color chart except where common terms of color definition are employed.
Original cultivar: Dendrobium hybrid ‘Anna Bride’.
Propagation: By division and micropropagation.
Plant:
   Shape.—Pseudobulb, cylidrical and upright.
   Size.—Pseudobulb about 50-70 cm high diameter about 2.0-2.5 cm at the age of 2.5-3.0 years.
   Color.—Pseudobulb near 143 C.
   Growth habit.—Resulting from branching of rhizome, the new pseudobulb emerges close to the old pseudobulb.
Foliage:
   Leaf shape.—Lanceolate with an acute apex, an obtuse base and entire margin.
   Size.—Length about 15-20 cm, width about 4.0-5.0 cm.
   Surface.—Smooth; upper near 141 A; lower near 143 B.
   Aspect.—Distichous alternate array on pseudobulb.
   Leaf sheath.—Length about 15- 20 cm; near 143 B.
Inflorescence: From apical and 3^{rd} to 4^{th} axillary buds of pseudobulb; 16 to 23 flowers.
   Raceme.—Erect, near 144A.
   Pedunole.—Length about 15-18 cm; diameter about 0.35 cm; near 144A.
   Rachis.—Length about 40-45 cm; diameter about 0.45 cm; near 144A.
   Flower.—Natural spread about 6.5-7.2 cm; vertical length about 5.3-5.8 cm; dorsal sepal about 1.8 to 2.0 cm wide, 3.7 to 3.9 cm long, near N 155 C, abaxil near 155 C, elliptic with cuspidate tip, entire; lateral sepals about 1.8 to 2.0 cm wide, 4.5 to 4.7 cm long, near N 155 C, abaxil near oblong with cuspidate tip, entire; petals about 3.4 to 3.5 cm wide, 3.7 to 3.9 cm long, near N 155 C, abaxil near 155 C, rhombic with acute tip, entire; labellum about 1.8 to 2.0 cm wide, 4.2 to 4.3 cm long, near 155 C, non-pubescence, trilobed rhombic, no appendage, labellar gill 5 rows, near 149 D, abaxil near 155 C; column about 0.63 cm long, diameter about 0.45 cm, near 150 C; spur about 1.8 cm long, near 157 D; pollinia 2 in number, about 0.1 cm wide and 0.2 cm long, near 7A; pedicel about 4.8-4.9 cm long, diameter 0.25 cm, near 145 D; nearly open bud about 1.5 cm wide, about 3.5 cm long, near 149 C.
   Vase life.—About 12-15 days in water.
   Disease and pest resistance.—none observed.

Plant of the Dendrobium ‘White Anna’ is upright and clumping. The inflorescence is long with 16-23 flowers, long lasting. Flower is white with light yellowish green gills. Petals overlap sepals.

1. A new and distinct plant of Dendrobium named ‘White Anna’ substantially as illustrated and described.
